(19) United States
(12) Patent Application Publication
(10) Pub. No.: US 2013/0017587 A1
(43) Pub. Date: Jan. 17, 2013

(54) Method for Recovery of Organic Components from Dilute Aqueous Solutions

(71) Applicants: Franz Nierlich, Marl (DE); Walter Burger-Kley, Klagenfurt (AT); Ilja Mikenberg, Bochum (DE); Bernhard Kneissel, Bonn (DE)

(72) Inventors: Franz Nierlich, Marl (DE); Walter Burger-Kley, Klagenfurt (AT); Ilja Mikenberg, Bochum (DE); Bernhard Kneissel, Bonn (DE)

(73) Assignee: Stratley AG, Koln (DE)

(21) Appl. No.: 13/515,796

(22) PCT Filed: Dec. 15, 2010
(86) PCT No.: PCT/EP10/69742

§ 371 Date: Jun. 13, 2012

(30) Foreign Application Priority Data
Dec. 15, 2009 (EP) 09179358.8

(51) Int. Cl. C12P 7/02; C12P 7/04; C12P 7/18; C12P 7/16

(52) U.S. Cl. 435/158; 435/155; 435/160; 435/157

(57) ABSTRACT

The present invention relates to a method for recovering an organic component from an aqueous medium such as a fermentation broth containing microorganism producing said organic component. The method includes increasing the activity of the organic component in the aqueous medium by increasing the concentration of at least one hydrophilic solute in the medium leading to salting-out of the organic component. The microorganisms are genetically modified to be capable of tolerating higher concentrations in the medium in comparison to their unmodified counterparts.

The present invention relates to a method for recovering an organic component from an aqueous medium such as a fermentation broth containing microorganisms producing said organic component. The method includes increasing the activity of the organic component in the aqueous medium by increasing the concentration of at least one hydrophilic solute in the medium leading to salting-out of the organic component. The microorganisms are genetically modified to be capable of tolerating higher concentrations of the hydrophilic solute in the medium in comparison to their unmodified counterparts.

### BACKGROUND OF THE INVENTION

The method of the present invention provides improved volumetric productivity for the fermentation and allows recovery of the fermentation product. The inventive method also allows for reduced energy use in the production due to increased concentration of the fermentation product by the simultaneous fermentation and recovery process which increases the quantity of fermentation product produced and recovered per quantity of fermentation broth. Thus, the invention allows for production and recovery of fermentation products at low capital and reduced operating costs.

Key parameters that control economic performance of a fermentation process are product concentration and volumetric productivity.

In some cases high concentrations of a fermentation product in a fermentation broth may have some toxic effects to microorganisms and/or inhibit a further fermentation process resulting in a highly diluted product and low volumetric productivity.

The low effective product concentration and volumetric productivity negatively impact several aspects of product economics, including equipment size and utility costs. As the product concentration decreases in the fermentation broth, recovery volumes of aqueous solutions are increased which results in higher capital costs and larger volumes of materials to process within the production plant.

The utilization of a recovery process to simultaneously remove fermentation products as they are produced, thus increasing product volumetric productivity and concentration may strongly influence product economics. For example, fermentation completed at twice the volumetric productivity will reduce fermentor cost by almost 50% for a large industrial fermentation facility. The fermentor capital cost and size reduction decreases depreciation and operating costs for the facility.

Similarly, using recovery processes in which product rich phases are formed and water rich phases are separated and discarded, the water load in the fermentation broth volume handled in downstream recovery and purification equipment is significantly reduced. For example, the doubling of product concentration in the recovered phase almost halves the amount of water to be processed for a given production volume reducing operating and capital costs.

Many technical approaches have been developed for the simultaneous removal of fermentation products from aqueous based fermentation media, including liquid/liquid extraction, membrane separations (e.g., pervaporation), adsorption and absorption. In the case described above where the fermentation product concentration in the fermentor stream is low, these approaches have significant impact on operating and capital costs, because of high energy consumption and expensive equipment making any commercial production unviable.

For example, today, the most widely used in sito recovery technique carried out at the industrial level is liquid-liquid extraction. In this process, an extraction solvent is mixed with the fermentation broth. Fermentation products are extracted into the extraction solvent and recovered by back-extraction into another extraction solvent or by distillation. Additionally to the above-described disadvantages, several problems are associated with liquid-liquid extraction, such as toxicity to the cells, the formation of an emulsion, loss of expensive extraction solvent, and the accumulation of microbial cells at the extractant and fermentation broth interphase.

Pervaporation is a membrane-based process that is used to remove solvents from fermentation broth by using a selective membrane. The liquids or solvents diffuse through a solid membrane leaving behind nutrients, sugar, and microbial cells. One problem commonly associated with pervaporation is economically providing and maintaining the chemical potential gradient across the membrane. Those pervaporation processes employing a vacuum pump or condenser to provide the necessary chemical potential gradient are energy-intensive and thus expensive to operate. As the concentration of the organic compound in the feed stream is reduced to low levels, the partial pressure of the vaporizable organic compound in the permeate stream must be kept even lower for permeation and therefore separation to take place. If a vacuum pump is used to maintain the difference in partial pressure of the organic compound in equilibrium with the liquid feed stream and the partial pressure of the vaporizable organic compound in the vapor-phase permeate, the pump must maintain a very high vacuum, thus incurring high capital and operating costs. Similarly, if a condenser is used, extremely low temperatures must be maintained, requiring a costly and complicated refrigeration system.

For commercial production, there is a need, therefore, for a low cost method to simultaneously remove fermentation products as they are produced to prevent the concentration of the toxic fermentation product from exceeding the tolerance level of the culture thus increasing volumetric productivity, as well as a method of recovering such fermentation product using phase separation to decrease processed water volume.

### PRIOR ART

US 2009/0171129 A1 describes a method for recovery of C₃- to C₆-alcohols (in the following also denoted “C₃₋₆-alcohols”) from dilute aqueous solutions, such as fermentation broths comprising a. increasing the activity of the C₃₋₆-alcohol in a portion of the fermentation broth to at least that of saturation of the C₃₋₆-alcohol in the portion; b. forming a C₃₋₆-alcohol-rich liquid phase and a water-rich liquid phase from the portion of the fermentation broth; and c. separating the C₃₋₆-alcohol-rich phase from the water-rich phase. The activity of the C₃₋₆-alcohol is increased, e.g. by salting-out, i.e. adding a hydrophilic solute to the aqueous solution.

Methods described in the prior art suffer from the drawback that microorganisms used for fermentation are often not tolerant to concentrations of hydrophilic solutes required for salting-out of the desired component. Thus, if not detrimental to the function of the prior art methods, productivity and cost effectiveness of such methods are at least substantially decreased.

### SUMMARY OF THE INVENTION

The technical problem of the present invention is therefore to further improve prior art methods as, e.g. described in US 2009/0171129 A1.

The solution to the above technical problem is provided by the embodiments of the present invention as characterized in the claims.

This invention relates to separation methods for recovery of organic components from dilute aqueous solutions, such as fermentation broths. Such methods provide improved volumetric productivity for the fermentation and allow recovery of the fermentation product. Such methods also allow for reduced energy use in the production due to increased concentration of the fermentation product by the simultaneous fermentation and recovery process which increases the quantity of fermentation product produced and recovered per quantity of fermentation broth. Thus, the invention allows for production and recovery of fermentation product at low capital and reduced operating costs.

In particular, the present invention provides a method for recovering an organic component from an aqueous medium, e.g. a fermentation broth, containing micoorganisms producing said organic component comprising the steps of:
(a) increasing the concentration of at least one hydrophilic solute in at least a portion of the aqueous medium so that the activity of the organic component in the portion of the aqueous medium is increased to at least the saturation of the organic component in the portion;
(b) forming a liquid phase rich in the organic component and a liquid water rich phase from the portion; and
(c) separating the liquid phase rich in the organic component from the water-rich phase;
   wherein the microorganisms are genetically modified to be capable of tolerating higher concentrations of the at least one hydrophilic solute in the portion of the aqueous medium than the unmodified microorganims.

Further subject matter of the invention relates to a method for producing an organic component comprising the steps of:
(A) culturing a microorganism in a fermentation medium to produce the organic component by said microorganism;
(B) recovering the organic product released by the microorganism into the fermentation medium from at least a portion of the fermentation medium by using the method for recovering an organic component from an aqueous solution as defined herein.

To date, a combination of a process to simultaneously remove fermentation products as they are produced by salting-out and a fermentation process with cells and organisms tolerant to high salt has never been reported.

### DESCRIPTION OF THE PREFERRED EMBODIMENTS

The term “fermentation” or “fermentation process” is defined as a process in which a microorganism is cultivated in a culture medium containing raw materials, such as feedstock and nutrients, wherein the microorganism converts raw materials, such as a feedstock, into products.

The term “organic component” may be any organic compound produced by microorganism and present in an aqueous solution, such as a fermentation broth.

The organic component may be an alcohol. Preferably, the alcohol is a C₃- to C₆-mono- or dialcohol, in particular propanol, butanol, pentanol, or hexanol, or a corresponding diol such as a propandiol, a butandiol, a pentandiol or a hexandiol. In some embodiments, the propanol may be 1-propanol or 2-propanol. In some embodiments, the butanol may be 1-butanol, 2-butanol, tert-butanol (2-methyl-2-propanol), or iso-butanol (2-methyl-1-propanol). In some embodiments, the pentanol may be 1-pentanol, 2-pentanol, 3-pentanol, 2-methyl-1-butanol, 3-methyl-1-butanol, 2-methyl-2-butanol, 3-methyl-2-butanol, or 2,2-dimethyl-1-propanol. In some embodiments, the hexanol may be 1-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 3,3-dimethyl-1-butanol, 2,2-dimethyl-1-butanol, 2,3-dimethyl-1-butanol, 2,3-dimethyl-2-butanol, 3,3-dimethyl-2-butanol, or 2 ethyl-1-butanol. In other preferred embodiments the diol may be selected from 1,2-propandiol, 1,3-propandiol, 1,2-butandiol, 1,3-butandiol, 2,3-butandiol and 1,4 butandiol.

In some embodiments the organic component may be an aldehyde.

According to the invention a hydrophilic solute such as for example sodium chloride is added to an aqueous solution such as fermentation broth in amount sufficient to cause salting out, which is separation of the solution into two immiscible phases; one phase is an aqueous sodium chloride solution and the other phase is an organic fermentation product solution. These two immiscible phases are physically separated, e.g. by gravity, to obtain a principally aqueous solution of hydrophilic solute with only minor proportions of the organic components and a principally organic solution with only a minor proportion of water.

The hydrophilic solute is preferably added to the entire fermentation broth in the fermentor to simultaneously remove fermentation products as they are produced to prevent the concentration of the toxic fermentation product from exceeding the tolerance level of the culture. The presence of various salts, e.g., sodium chloride, or other dissolved components can seriously inhibit growth of organisms exposed to such conditions. In organisms such as yeast or bacteria, high salt medium can cause dehydration of the cells, as well as interfere with metabolism, causing growth inhibition or cell destruction. The provision of salt-tolerant organisms is therefore useful in allowing growth of the organisms under adverse conditions that normally would not support a useful level of growth, or not support growth at all.

According to an embodiment of the invention, increasing the activity of the organic component may comprise adding a hydrophilic solute to the aqueous solution. In some embodiments in which the aqueous solution is a fermentation broth, the hydrophilic solute is preferably added to the entire fermentation broth in the fermentor. Reference to adding a hydrophilic solute can refer to increasing the concentration of a hydrophilic solute already existing in the portion of the solution or to addition of a hydrophilic solute that was not previously in the solution. Such increase in concentration may be done by external addition. Alternatively, or additionally, increasing concentration may also be conducted by in situ treatment of the solution, such as by hydrolyzing a solute already existing in the solution, e.g. hydrolyzing proteins to add amino acids to the solution, hydrolyzing starch or cellulose to add glucose to the solution and/or hydrolyzing hemicellulose to add pentoses to the solution. According to another preferred embodiment, the hydrophilic solute may be one that has a nutritional value and optionally ends up in a fermentation coproduct stream, such as distillers dried grains and solubles (DDGS). In addition or alternatively, the hydrophilic solute can be fermentable and can be transferred with the water-rich liquid phase to the fermentor. Generally, the hydrophilic solute is selected from salts, amino acids, water-soluble solvents, sugars and combinations thereof.

The method further includes the step of forming a phase rich in the organic component such as forming a C₃₋₆-alcohol-rich liquid phase and a water-rich liquid phase from the portion of the aqueous solution which has been treated to increase the activity of the organic component, e.g. a C₃₋₆-alcohol. As used herein, the term “organic component-rich phase” (e.g. an “alcohol-rich liquid phase”) means a liquid phase wherein the organic component-to-water ratio is greater than that in the portion of the starting aqueous solution. The term “water-rich liquid phase” means a liquid phase wherein the water-to-organic component ratio is greater than that of the organic component-rich liquid phase. The water-rich phase may also be referred to as organic component-lean phase, e.g. an alcohol-lean phase. The step of forming the two phases can be active. For example, in some embodiments, the step of forming may comprise condensing a distilled vapor phase that forms two phases after condensation. Alternatively or in addition, chilling or cooling the treated portion of the aqueous solution can result in the formation of the two phases. Other steps for actively forming the two phases can include using equipment shaped to promote the separation of phases. Separation of the phases can be accomplished in various unit operations including liquid-liquid separators comprising a liquid/liquid separator utilizing specific gravity differences between the phases and a water boot, g-force separation as in a centrifuge, or centrifugal liquid-liquid separators. Also suitable are settlers as in mixer-settler units used for solvent extraction processes. In some embodiments the step of forming is passive and may simply be a natural consequence of the previous step of increasing the activity of the organic component, preferably a C₃₋₆-alcohol, to at least that of saturation.

Thus, in the organic component-rich liquid phase, the ratio of the concentration of the organic component with respect to the water is effectively greater than in the starting portion. In the water-rich phase, the ratio of concentration of the organic component with respect to water is effectively less than in the organic component-rich liquid phase. The water-rich phase may also be referred to as the organic component-poor phase (e.g. an alcohol-poor phase).

Preferred embodiments of the present invention relate to the recovery of C₃₋₆-alcohol from dilute aqueous solutions containing micoorganisms producing the alcohol as defined herein. With respect to specific C₃₋₆-alcohols, typical concentrations in the alcohol-rich phase can be given as follows: in some of such embodiments the alcohol is propanol and the weight ratio of propanol to water in the alcohol-rich phase is greater than about 0.2, greater than about 0.5, or greater than about 1. In other embodiments, the C₃₋₆-alcohol is butanol and the ratio of butanol to water in the alcohol-rich phase is greater than about 1, greater than about 2, or greater than about 8. In further embodiments, the C₃₋₆-alcohol is pentanol and the ratio of pentanol to water in the alcohol-rich phase is greater than about 4, greater than about 6, or greater than about 10.

The concentration factor or enrichment factor for a given phase can be expressed as the ratio of organic compound (e.g. an alcohol) to water in that phase divided by the ratio of organic component to water in the dilute aqueous solution. Thus, for example, the concentration or enrichment factor for the organic component-rich phase may be expressed as the ratio of organic component/water in the organic component-rich phase divided by that ratio in the aqueous dilute solution. In preferred embodiments, the ratio of the organic component (such as a C₃₋₆-alcohol) to water in the organic component-rich phase is greater than the ratio of the organic component (e.g. a C₃₋₆-alcohol) to water in the starting aqueous solution, e.g. a fermentation broth, by at least about 5 fold, at least about 25 fold, at least about 50 fold, at least about 100 fold, or at least about 300 fold.

The method of the invention further includes separating the organic component-rich liquid phase (e.g. a C₃₋₆-alcohol-rich phase) from the water-rich phase. Separating the two phases refers to physical separation of the two phases and can include removing, skimming, pouring out, decanting or otherwise transferring one phase from another and may be accomplished by any means known in the art for separation of liquid phases.

According to preferred embodiments of the invention, the organic component such as an alcohol, preferably a C₃- to C₆-mono-alcohol or -diol as outlined above, is further purified from the liquid phase rich in the organic component obtained in step (c) (herein after also denoted as step (d)). In various embodiments, the step (d) may include the step of distillation, dialysis, water adsorption, extraction of the organic component by solvent extraction, contact with a hydrocarbon liquid that is immiscible in water or contact with a hydrophilic compound. This step may produce two phases including a first phase containing the organic compound such as a C₃₋₆-alcohol and water and a second phase containing the organic component such as a C₃₋₆-alcohol, wherein the ratio of water to organic component (e.g. a C₃₋₆-alcohol) in the second phase is less than in the first phase. In various embodiments, the second phase may contain at least about 90% by weight alcohol, at least about 95% by weight alcohol or at least about 99% by weight alcohol.

Distillation is a preferred measure to further purify the organic component from the liquid phase rich in the organic component in step (c). In some embodiments, distilling is conducted at below atmospheric pressure and at a temperature of between about 20° C. and about 95° C. In some embodiments, the step of distilling is conducted at a pressure of from about 0.025 bar to about 10 bar. According to preferred embodiments of the invention, the step of processing the liquid phase rich in the desired organic component such as a C₃- to C₆-alcohol may include distilling substantially pure C₃₋₆-alcohol from the C₃₋₆-alcohol-rich phase.

In some embodiments, processing may include distilling an azeotrope of the C₃₋₆-alcohol from the C₃₋₆-alcohol-rich phase. In some embodiments, processing may further include contacting the C₃₋₆-alcohol-rich phase with a C₃₋₆-alcohol-selective adsorbent. In some embodiments, processing may include converting C₃₋₆-alcohol in the C₃₋₆-alcohol-rich phase to an olefin. In some embodiments, processing may include combining the C₃₋₆-alcohol-rich phase with a hydrocarbon liquid that is immiscible in water. In some embodiments, the combination may form a single uniform phase. In some embodiments, the combination may form a light phase and a heavy phase and the ratio of alcohol to water in the light phase may be greater than the ratio in the heavy phase.

Further general guidance with respect to steps (a) to (c) and, optionally, (d) can be found in the prior art, specifically for C₃- to C₆-alcohols, e.g. in respective sections of US 2009/0171129 A1, the corresponding contents of which is incorporated herewith into the present description in its entirety by reference.

The invention provides microorganisms which produce an organic product by fermentation with limited water solubility. The microorganisms comprise a genetic modification that results in enhanced tolerance against a hydrophilic solute used to increase the activity of the organic component produced by the microorganisms. The genetic modification preferably leads to intracellular accumulation of at least one small molecule (osmolyte) in the cytoplasm to counteract the external osmotic pressure as compared with cells that lack the genetic modification (i.e. unmodified microorganisms in respect of this modification). Host cells of the invention may produce the fermentation product naturally or may be engineered to do so via an engineered metabolic pathway.

Such genetic modification and resulting tolerance of osmotic pressure can be obtained in a variety of different cells. Any suitable host cell may be used in the practice of the present invention. For example, the host cell can be a genetically modified host microorganism in which nucleic acid molecules have been inserted, deleted or modified (i.e., mutated; e.g., by insertion, deletion, substitution, and/or inversion of one or more nucleotides). Typical microorganisms useful in the method of the present invention are bacteria and yeast.

Examples of bacterial microorganisms useful in the context of the present invention include but are not limited to: Bacillus subtilis, Bacillus amyloliquefacines, Brevibacterium ammoniagenes, Brevibacterium immariophilum, Clostridium beigerinckii, Clostridium acetobutylicum, Clostridium butylicum, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, Staphylococcus aureus, and the like.

Examples of yeast microorganisms useful in the context of the present invention include but are not limited to: Aspergillus nidulans, Aspergillus niger, Aspergillus oryzae, Candida albicans, Chrysosporium lucknowense, Fusarium graminearum, Fusarium venenatum, Kluyveromyces lactis, Neurospora crassa, Pichia angusta, Pichia finlandica, Pichia kodamae, Pichia membranaefaciens, Pichia methanolica, Pichia opuntiae, Pichia pastoris, Pichia pijperi, Pichia quercuum, Pichia salictaria, Pichia thermotolerans, Pichia trehalophila, Pichia stipitis, Streptomyces ambofaciens, Streptomyces aureofaciens, Streptomyces aureus, Saccharomyces bayanus, Saccharomyces boulardi, Saccharomyces cerevisiae, Streptomyces fungicidicus, Streptomyces griseochromogenes, Streptomyces griseus, Streptomyces lividans, Streptomyces olivogriseus, Streptomyces rameus, Streptomyces tanashiensis, Streptomyces vinaceus, and Trichoderma reesei.

Thus, in various embodiments, the cell is a yeast cell which may be selected from the species as outlined above, preferably a Saccharomyces cell, most preferably a Saccharomyces cerevisiae cell. Likewise, as already outlined above, the cell may be from a bacterial species, preferably Escherichia coli. Other bacterial species useful as genetically modified microorganisms in the context of the present invention are derived from hydrocarbonoclastic bacteria (HCB) such as representatives of the genera Alcanivorax (e.g. A. borkumensis), Cycloclasticus, Marinobacter, Neptunomonas, Oleiphilus, Oleispira and Thalassolitus. In preferred embodiments, the cell is in a cell culture, preferably in a population of such cells. Preferably, the cell culture is a liquid culture. In preferred embodiments, the cell culture is a high density cell culture.

The accumulation of organic solutes is a prerequisite for osmotic adjustment of all microorganisms: to adjust to lower water activities of the environment and the resulting decrease in cytoplasmic water, microorganisms must accumulate intracellular ions or organic solutes to reestablish the cell turgor pressure and/or cell volume and, at the same time, preserve enzyme activity.

Microorganisms have developed two main strategies for osmotic adjustment. One strategy relies on the selective influx of K⁺ from the environment to, sometimes extremely, high levels and is known as the ‘salt-in-the-cytoplasm’ type of osmotic adaptation (Galinski E. A., Advances in Microbial Physiology 37:272-328 (1995); da Costa, M. S. et al., Advances in Biochemical Engineering/Biotechnology 61:117-153 (1998); Roeβler, M. and Müller, V., Environmental Microbiology 3: 743-754 (2001)). This type of osmotic adjustment occurs in the extremely halophilic archaea of the family Halobacteriaceae, the anaerobic moderately halophilic bacteria of the Order Halanaerobiales (Oren, A. Microbiology and Molecular Biology Reviews 63: 334-348 (1999)) and the extremely halophilic bacterium Salinibacter ruber (Anton, J. et al., International Journal of Systematic and Evolutionary Microbiology 52: 485-491 (1999); Oren, A. and Mana, L., Extremophiles 6: 217-223 (2002)).

The majority of microorganisms have not, however, undergone extensive genetic alterations as a prerequisite for adaptation to a saline environment and, the intracellular macromolecules are generally sensitive to high levels of inorganic ions. These organisms favour the accumulation of specific small-molecular weight compounds, known as compatible solutes or osmolytes (Brown, A. D., Bacteriological Reviews 40: 803-846 (1976), Brown, A. D., Microbial Water Stress Physiology: Principles and Perspectives. Chichester: John Wiley & Sons (1990); Ventosa, A. et al., Microbiology and Molecular Biology Reviews 62: 504-544 (1998)). Compatible solutes can also be taken up from the environment, if present or, they can be synthesized de nova. The most common compatible solutes of microorganisms are neutral or zwitterionic and include amino acids and amino acid derivatives, sugars, sugar derivatives (heterosides) and polyols, betaines and the ectoines (da Costa, M. S. et al., Advances in Biochemical Engineering/Biotechnology 61: 118-153 (1998)). Some are widespread in microorganisms, namely trehalose, glycine betaine and α-glutamate, while others are restricted to a few organisms. Polyols, for example, are widespread among fungi and algae but are very rare in bacteria and unknown in archaea. Ectoine and hydroxyectoine are examples of compatible solutes found only in bacteria.

According to preferred embodiments, the osmolyte accumulated by the microorganisms may be selected from trehalose, glycine betaine, proline, glycerol, ectoine and hydroxyectoine.

Enhanced accumulation of such osmolytes is preferably obtained by genetic modification of one or more biochemical pathways in the microorganism used for producing the desired organic component.

The genetic modification of the microorganisms according to the present invention relies in one or more proteins involved in the production and/or processing and/or cellular transport (export, import) of the osmolyte or one or more of its precursors.

Generally, the modified microorganisms according to the invention carry a gene or genetic construct allowing the (over)expression of one or more proteins involved in the above-mentioned pathways. Molecular biological operations required for assembling useful genetic vehicles (such as plasmids, viruses), transfection and expression of desired constructs are known in the art (see, e.g. Ausubel et al. (eds.) Current Protocols in Molecular Biology, John Wiley & Sons, New York, 2001-2009).

The term “gene” or “genetic construct” refers to a nucleic acid fragment that is capable of being expressed as a specific protein, optionally including regulatory sequences preceding (5′ non-coding sequences) and following (3′ non-coding sequences) the coding sequence. “Native gene” refers to a gene as found in nature with its own regulatory sequences. “Recombinant gene” refers to any gene that is not a native gene, comprising regulatory and coding sequences that are not found together in nature. Accordingly, a recombinant gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or recombinant genes. A “transgene” is a gene that has been introduced into the genome by a transformation procedure.

The term “expression”, as used herein, refers to the transcription and stable accumulation of sense (mRNA) or antisense RNA derived from the nucleic acid fragment of the invention. Expression may also refer to translation of mRNA into a polypeptide.

Generally, the nomenclature and the laboratory procedures in recombinant DNA technology described herein are well known to the person skilled in the art. Standard techniques are used for cloning, DNA and RNA isolation, amplification and purification. Generally, enzymatic reactions involving DNA ligase, DNA polymerase, restriction endonucleases and the like are performed according to the manufacturer's specifications. These techniques and various other techniques are generally performed according to Sambrook et al., Molecular Cloning—A Laboratory Manual, Cold Spring Harbor Laboratory, Cold Spring Harbor, N.Y. (1989).

Materials and methods suitable for the routine maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds.), American Society for Microbiology, Washington, D.C. (1994)).

As used herein the term “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

For many applications such as introduction of a heterologous gene, coding sequence, or regulatory sequence, it is often necessary to introduce nucleic acid sequences into the respective cells. A number of such methods are known and can be utilized, with the specific selection depending on the particular type of cells.

For transformation of E. coli strains electrocompetent cells can be prepared prepared as follows: E. coli are grown in SOB-medium (Sambrook, J., Russel, D. W. Molecular Cloning, A Laboratory Manual. 3 ed. 2001, Cold Spring Harbor, N.Y.: Cold Spring Harbor Laboratory Press) to an OD600 of about 0.6 to 0.8. The culture is concentrated 100-fold, washed once with ice cold water and 3 times with ice cold 10% glycerol. The cells are then resuspended in 150 μL of ice-cold 10% glycerol and aliquoted into 50 μL portions. These aliquots can be used immediately for standard transformation or stored at −80° C. These cells are transformed with the desired plasmid(s) via electroporation. After electroporation, SOC medium (Sambrook, J., Russel, D. W. Molecular Cloning, A Laboratory Manual. 3 ed. 2001, Cold Spring Harbor, N.Y.: Cold Spring Harbor Laboratory Press) is immediately added to the cells. After incubation for an hour at 37° C. the cells are plated onto LB-plates containing the appropriate antibiotics and incubated overnight at 37° C.

Yeast cells can, for example, be transformed by converting yeast cells into protoplasts, e.g., using zymolyase, lyticase, or glusulase, followed by addition of the nucleic acid and polyethylene glycol (PEG). The PEG-treated protoplasts are then regenerated by culturing in a growth medium, e.g., under selective conditions (see, e.g., Beggs, Nature 275:104-108 (1978); and Hinnen et al., Proc. Natl. Acad. Sci. USA 75:1929-1933 (1978)). Another method does not involve removal of the cell wall, instead utilizing treatment with lithium chloride or acetate and PEG and then growth on selective media (see, e.g., Ito et al., J. Bact. 153:163-168 (1983)). A variety of methods for yeast transformation, integration of genes into the yeast genome, and growth and selection of yeast strains is described in Current Protocols in Molecular Biology, Vols. 1 and 2, Ausubel et al., eds., John Wiley & Sons, New York (1997).

The terms “plasmid” and “vector” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequences into a cell. “Recombinant vector” refers to a specific vector containing a foreign gene and having elements in addition to the foreign gene that facilitates transformation of a particular host cell.

Recombinant organisms containing the necessary genes that will encode the enzymatic pathway for the conversion of a fermentable carbon substrate to a desired organic product may be constructed using techniques well known in the art; see, for example, US-A-20070092957, US-A-20090239275, US-A-20090155870, US-A-20090155870, WO-A-2009/103533, US-A-20090246842.

A yeast strain of the present invention which is genetically modified for increased production of trehalose has improved tolerance to different salts. The tolerance of strains may be assessed by assaying their growth in concentrations of different salts, including sodium chloride, that are detrimental to growth of the parental (prior to genetic modification) strains.

Fermentation media of use in the present invention contain suitable carbon substrates. Suitable substrates include, but are not limited to, monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt.

In addition to an appropriate carbon source, fermentation media typically contain suitable minerals, salts, cofactors, buffers and other components, known to those skilled in the art, suitable for the growth of the cultures and promotion of the enzymatic pathway necessary for production of the desired organic compound.

For cell culture, cells are typically grown at a temperature in the range of about 20° C. to about 37° C. in an appropriate medium. Suitable growth media useful in the present invention may be common commercially prepared media such as broth that includes yeast nitrogen base, ammonium sulfate, and dextrose (as the carbon/energy source) or YPD Medium, a blend of peptone, yeast extract, and dextrose in optimal proportions for growing most Saccharomyces cerevisiae strains. Other defined or synthetic growth media may also be used and the appropriate medium for growth of the particular microorganism will be known by one skilled in the art of microbiology and/or fermentation science.

Suitable pH ranges for the fermentation are typically from about pH 3.0 to about pH 7.5, wherein from about pH 4.5.0 to about pH 6.5 is preferred as the initial condition.

The amount of the desired product, e.g. butanol, produced in the fermentation medium can be determined using a number of methods known in the art, for example, high performance liquid chromatography (HPLC) or gas chromatography (GC).

An example of genetic modification useful in the context in the present invention is described in U.S. Pat. No. 7,005,291 relating to a method for the production of glycerol from a recombinant organism comprising: transforming a suitable host cell with an expression cassette comprising either one or both of (a) a gene encoding a protein having glycerol-3-phosphate dehydrogenase (G3PDH) activity and (b) a gene encoding a protein having glycerol-3-phosphate phosphatase activity. This genetic modification results in enhanced intracellular accumulation of glycerol. With regard to details of the production of corresponding modified microorganisms it is referred to U.S. Pat. No. 7,005,291.

The terms “glycerol-3-phosphate dehydrogenase” and “G3PDH” refer to a polypeptide responsible for an enzyme activity that catalyzes the conversion of dihydroxyacetone phosphate (DHAP) to glycerol-3-phosphate (G3P). In vivo G3PDH may be NADH; NADPH; or FAD-dependent. The NADH-dependent enzyme (EC 1.1.1.8) is encoded, for example, by several genes including GPD1 (GenBank Z74071x2), or GPD2 (GenBank Z35169x1), or GPD3 (GenBank G984182), or DAR1 (GenBank Z74071x2). The NADPH-dependent enzyme (EC 1.1.1.94) is encoded by gpsA (GenBank U321643, (cds 197911-196892) G466746 and L45246). The FAD-dependent enzyme (EC 1.1.99.5) is encoded by GUT2 (GenBank Z47047x23), or glpD (GenBank G147838), or glpABC (GenBank M20938).

The terms “glycerol-3-phosphatase”, “sn-glycerol-3-phosphatase”, or “d,1-glycerol phosphatase”, and “G3P phosphatase” refer to a polypeptide responsible for an enzyme activity that catalyzes the conversion of glycerol-3-phosphate and water to glycerol and inorganic phosphate. G3P phosphatase is encoded, for example, by GPP1 (GenBank Z47047x125), or GPP2 (GenBank U18813x11).

The terms “GPP1”, “RHR2” and “YIL053W” are used interchangeably and refer to a gene that encodes a cytosolic glycerol-3-phosphatase and is characterized by the amino acid sequence given in SEQ ID NO: 7.

The terms “GPP2”, “HOR2” and “YER062C” are used interchangeably and refer to a gene that encodes a further cytosolic glycerol-3-phosphatase and is characterized by the amino acid sequence given as SEQ ID NO: 8.

Further genes useful in the present invention are genes involved in trehalose metabolism. Examples are genes coding for proteins with trehalose-6-phosphate synthase function such as corresponding enzymes from yeast, in particular Sacharomyces cerevisiae. Particular useful representatives of such enzymes (and their coding genes) are Tps1p, Tps2p, Tps3p and Tsl1p.

Genetically modified microorganisms useful in the context of the present invention expressing inter alia Tps1p are described in more detail in U.S. Pat. No. 5,422,254 and with regard to details of the production of corresponding modified microorganisms it is referred to this prior art document. Tps1p is a synthase subunit of the trehalose-6-phosphate synthase/phosphatase complex, which synthesizes the storage carbohydrate trehalose. In its natural context, expression of this protein is induced by stress conditions (e.g. osmotic stress).

Tps2p is a phosphatase subunit of the yeast trehalose-6-phosphate synthase/phosphatase complex, which synthesizes the storage carbohydrate trehalose. Its expression is induced by stress conditions (e.g. osmotic stress).

Tps3p is a regulatory subunit of the yeast trehalose-6-phosphate synthase/phosphatase complex, which synthesizes the storage carbohydrate trehalose; expression is induced by stress conditions (e.g. osmotic stress).

Tsl1p is a large subunit of the yeast trehalose 6-phosphate synthase (Tps1p)/phosphatase (Tps2p) complex, which converts uridine-5′-diphosphoglucose and glucose 6-phosphate to trehalose.

Further genes involved in trehalose metabolism are known from bacteria, in particular E. coli, such as trehalose-6-phosphate synthase genes like otsA and otsB.

Further genes useful in the present invention are genes involved in ectoine metabolism. Examples are genes coding for proteins having a role in ectoine biosynthesis such as L-2,4-diaminobutyric acid acetyltransferase (DABA acetyltransferase; catalyzes the acetylation of L-2,4-diaminobutyrate (DABA) to gamma-N-acetyl-alpha,gamma-diaminobutyric acid (ADABA) with acetyl coenzyme A), diaminobutyrate-2-oxoglutarate transaminase (catalyzes reversively the conversion of L-aspartate beta-semialdehyde (ASA) to L-2,4-diaminobutyrate (DABA) by transamination with L-glutamate) and L-ectoine synthase (Catalyzes the circularization of gamma-N-acetyl-alpha,gamma-diaminobutyric acid (ADABA). Ectoine (1,4,5,6-tetrahydro-2-methyl-4-pyrimidine carboxylic acid) is an excellent osmoprotectant.

Ectoine biosynthetic genes are known, e.g. from halobacteria such as Marinococcus halophilus. Specific examples include ectA, ectB and ectC; for further details see “Characterization of genes for the biosynthesis of the compatible solute ectoine from Marinococcus halophilus and osmoregulated expression in Escherichia coli.” Louis P., Galinski E. A.; Microbiology 143:1141-1149 (1997).

Other genes useful in the context of the present invention are involved in transport mechanisms, e.g. various ATP-dependent transport proteins and K⁺-syn- and antiporter proteins leading to increased cellular uptake of osmoprotecting compounds. Specific examples of such genes are known, e.g. from E. coli and include ProV, ProW, ProX and ProP. Proteins expressed from ProV, ProW and ProX genes lead to an intracellular accumulation of glycine betaine, proline and/or ectoine and are components of a multicomponent binding-protein-dependent transport system (the proU transporter) which serves as the glycine betaine/L-proline transporter. ProP encodes an osmoprotectant/proton symporter capable of transporting proline and glycine betaine, and mediates the uptake of osmoprotectants to adapt to increases in osmotic pressure.

Yet another class of genetic constructs useful for modifying microorganisms according to the present invention relates to genes involved in glycine betaine biosynthesis from choline. Examples are genes coding for choline synthase or betaine-aldehyde dehydrogenase. Representatives are known, e.g. from E. coli and include betA and betB.

The following table lists particular examples of proteins expressed in the micoorganisms useful in the inventive method.

| | | SEQ ID NO: of encoded amino |
|---|---|---|
| | | acid sequence according to |
| Organism | Gene | appended sequence listing |
| Sacharomyces | Tps1 | 1 |
| cerevisiae | | |
| Sacharomyces | Tps2 | 2 |
| cerevisiae | | |
| Sacharomyces | Tps3 | 3 |
| cerevisiae | | |
| Sacharomyces | Tsl1 | 4 |
| cerevisiae | | |
| Sacharomyces | GPD1 | 5 |
| cerevisiae | | |
| Sacharomyces | GPD2 | 6 |
| cerevisiae | | |
| Sacharomyces | HOR2 | 7 |
| cerevisiae | | |
| Sacharomyces | RHR2 | 8 |
| cerevisiae | | |
| Marinococcus | ectA | 9 |
| halophilus | | |
| Marinococcus | ectB | 10 |
| halophilus | | |
| Marinococcus | ectC | 11 |
| halophilus | | |
| Escherichia coli | otsA | 12 |
| Escherichia coli | otsB | 13 |
| Escherichia coli | ProP | 14 |
| Escherichia coli | betA | 15 |

The present invention is further illustrated by the following non-limiting examples:

### EXAMPLES

### Example 1

### Construction of n-Butanol Producing Yeast Strains Tolerating Higher Concentrations of NaCl in Medium

The Example section below, which describes the cloning and overexpression of in trehalose metabolism involved gene Tps1p in S. cerevisiae, is exemplary of a general approach for genetic modification of a biochemical pathways in the microorganism used for producing the desired organic component. This example illustrates as to how genes, e.g. those listed in the above Tab. 1, can be used to construct recombinant vectors for transferring gene capable of conferring salt tolerance to transgenic microorganisms.

This example provides a recombinant yeast host cell having the following characteristics: 1) the yeast host produces butanol when grown in a medium containing a carbon substrate; 2) the yeast host cell comprises at least one genetic modification which increases the tolerance to at least one hydrophilic solute in the medium compared to wild type cells.

Construction of n-Butanol Producing S. cerevisiae Strain

n-butanol producing yeast strains are constructed as described previously (Steen E J, Chan R, Prasad N, Myers S, Petzold C J, Redding A, Ouellet M, Keasling J D: Metabolic engineering of Saccharomyces cerevisiae for the production of n-butanol. Microbial Cell Factories 2008, 7:36).

Clostridium beijerinckii NCIMB 8052 is purchased from ATCC, catalog number 51743. C. beijerinckii genes are cloned from genomic DNA: thl, encodes thiolase; hbd, 3-hydroxybutyryl-CoA dehydrogenase; crt, crotonase; bcd, butyryl-CoA dehydrogenase; etfA & etfB, two-electron transferring flavoproteins A & B; and AdhE2 butyraldehyde dehydrogenase. E. coli strains DH10B and DH5á are used for bacterial transformation and plasmid amplification in the construction of the expression plasmids. The strains are cultivated at 37° C. in Luria-Bertani medium with 100 mg ampicillin. S. cerevisiae strain BY4742, a derivative of S288C, is used as the parent strain for all yeast strains. This strain is grown in rich YPD medium at 30° C.

Plasmids are constructed by the SLIC method, as previously described (Li M Z, Elledge S J: Harnessing homologous recombination in vitro to generate recombinant DNA via SLIC. Nat Methods 2007, 4(3):251-6.) They contain the 2μ origin of replication, LEU or HIS genes for selection, the GAL1 or GAL10 promoters, and the CYC1, ADH1, or PGK1 transcription terminators. The first three genes of the n-butanol pathway are integrated into the plasmid pESC-LEU (Stratagene) and the last four genes are placed on the plasmid pESC-HIS (Stratagene). All genes are PCR amplified with Phusion polymerase (New England Biolabs). Primers are designed to have 30-bp flanking regions homologous to the plasmid insertion regions, either the GAL1 or GAL10 promoter and the CYC1, ADH1, or PGK1 terminator.

n-butanol producing yeast strains are constructed by the co-transformation of the plasmids as outlined above into Saccharomyces cerevisiae BY4743 (ATCC 201390) followed by selection on SD-LEU-HIS plates. Yeast transformation is performed by a lithium acetate method (Gietz, R. D., and R. A. Woods. 2002. Transformation of yeast by lithium acetate/single-stranded carrier DNA/polyethylene glycol method. Methods Enzymol. 350:87-96). Yeast cells are grown overnight in YPD, diluted 1:10 in 10 ml of fresh YPD, and allowed to grow 5 h at 28° C. with shaking. The cells are then collected by centrifugation, washed once with sterile water, and suspended in 100 μl of sterile water. Fifty microliters of the cell suspension are then mixed with 115 μl of 60% polyethylene glycol 3350, 5 μl of 4 M lithium acetate, 15 μl of sterile water, 10 μl of 10 mg/ml carrier DNA, and 5 μl of PCR product. The mixture is vortexed for 30 s, incubated at 42° C. for 40 min, and spread on appropriate plates.

Construction of n-Butanol Producing Yeast Strains Tolerating Higher Concentrations of NaCl in Medium

The Tps1p gene is cloned from genomic DNA prepared from the S. cerevisiae S288C strain. The Tps1p gene is inserted into the pESC-URA (Stratagene) plasmid. The gene is PCR amplified using Phusion polymerase (New England Biolabs). Primers are designed to have 30-bp flanking regions homologous to the plasmid insertion regions, either the GAL1 or GAL10 promoter and the CYC1 or ADH1 terminator.

n-Butanol producing yeast strains tolerating higher concentrations of salts in medium, including NaCl, are constructed by the transformation of the pESC-URA-Tps1p plasmid into cells of Saccharomyces cerevisiae BY4743 (ATCC 201390) carrying the pESC-LEU/pESC-HIS plasmids for expression of n-butanol pathway genes as described above followed by selection on SD-LEU-HIS-URA plates.

In comparison to control cells, yeast cultures overexpressing Tps1p gene show (depending on the strain) difference in viability of 2 to 3 log units.

### Example 2

### Phase Separation of Butanol in the Fermentation Medium by Addition of Hydrophilic Compound

This example illustrates the induction of phase separation of butanol in the fermentation medium of cells prepared according to Example 1 by addition of a hydrophilic compound.

Several yeast fermentation media are prepared for each salt, differing in their salt concentrations. Cells are routinely grown with shaking (160 rpm) at 30° C. in medium supplemented with galactose. During fermentation, phase separation can be observed forming an upper, butanol-rich phase (light phase) and a lower, alcohol-lean phase (heavy phase). The phase ratio between the aqueous solution and the solvent differ from one case to the other. Both phases are analyzed for alcohol and water content.

For n-butanol detection, 2 ml ethyl acetate containing n-pentanol (0.005% v/v), an internal standard, is added to the 10 ml sample and vortexed for 1 min. The ethyl acetate is then recovered and applied to a Thermo Trace Ultra gas chromatograph (GC) equipped with a Triplus AS autosampler and a TR-WAXMS column (Thermo Scientific). The samples are run on the GC according to the following program: initial temperature, 40° C. for 1.2 min, ramped to 130° C. at 25° C./min, ramped to 220° C. at 35° C./min. Final quantification analysis is carried out using the Xcalibur software.

The water content of the organic phases is determined by the Karl-Fischer method. The distribution coefficient of the alcohol is calculated for each experiment by dividing the alcohol concentration in the light phase by the concentration in the heavy phase. All experiments are carried out at 30° C.

The results are summarized in the following Table 2.

TABLE 2

| | Butanol in light | Butanol in heavy | Distribution |
|---|---|---|---|
| Hydrophilic solute | phase (% w/w) | phase (% w/w) | coefficient |
| Natrium chloride | 94 | 2.8 | 33.6 |
| (8% w/w) | | | |
| Calcium chloride | 96 | 2.2 | 43.6 |
| (8% w/W) | | | |

The results show that butanol separation during fermantaion can be reached, if either natrium or calcium chloride is present in the fermentation medium.

### REFERENCES CITED

Gietz, R. D., and R. A. Woods. 2002. Transformation of yeast by lithium acetate/single-stranded carrier DNA/polyethylene glycol method. Methods Enzymol. 350:87-96.
Steen E J, Chan R, Prasad N, Myers S, Petzold C J, Redding A, Ouellet M, Keasling J D: Metabolic engineering of Saccharomyces cerevisiae for the production of n-butanol. Microbial Cell Factories 2008, 7:36.
Li M Z, Elledge S J: Harnessing homologous recombination in vitro to generate recombinant DNA via SLIC. Nat Methods 2007, 4(3):251-6.

What is claimed is:

1. A method for recovering an organic component from an aqueous medium containing micoorganisms producing said organic component comprising the steps of
(a) increasing the concentration of at least one hydrophilic solute in at least a portion of the aqueous medium so that the activity of the organic component in the portion of the aqueous medium is increased to at least the saturation of the organic component in the portion;
(b) forming a liquid phase rich in the organic component and a liquid water rich phase from the portion; and
(c) separating the liquid phase rich in the organic component from the water-rich phase;
wherein the microorganisms are genetically modified to be capable of tolerating higher concentrations of the at least one hydrophilic solute in the portion of the aqueous medium than the unmodified microorganims.

2. The method of claim 1 wherein the aqueous medium is a fermentation broth.

3. The method of claim 1 wherein the organic component is an alcohol.

4. The method of claim 3 wherein the alcohol is a C₃- to C₆-mono- or -dialcohol.

5. The method of claim 4 wherein the C₃- to C₆-mono-alcohol is selected from the group consisting of 1-butanol, 2-butanol, tert-butanol, iso-butanol, 1-pentanol, 2-pentanol, 3-pentanol, 2-methyl-1-butanol, 3-methyl-1-butanol, 2-methyl-2-butanol, 3-methyl-2-butanol, 2,2-dimethyl-1-propanol, 1-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 3,3-dimethyl-1-butanol, 2,2-dimethyl-1-butanol, 2,3-dimethyl-1-butanol, 2,3-dimethyl-2-butanol, 3,3-dimethyl-2-butanol and 2 ethyl-1-butanol.

6. The method of claim 4 wherein the C₃- to C₆-dialcohol is selected from the group consisting of 1,2-propandiol, 1,3-propandiol, 1,2-butandiol, 1,3-butandiol, 2,3-butandiol and 1,4 butandiol.

7. The method of claim 1 wherein the hydrophilic solute is selected from the group consisting of salts, amino acids, water-soluble solvents, sugars and combinations thereof.

8. The method of claim 1 wherein the microorganisms have a genetic modification resulting in enhanced accumulation of at least one osmolyte in the cytoplasm of the microorganisms.

9. The method of claim 8 wherein the osmolyte is selected from the group consisting of trehalose, glycine betaine, proline, glycerol and ectoine.

10. The method of claim 9 wherein the microorgansims are modified to express a protein from one or more genes selected from the group consisting of Tps1, Tps2, Tps3, Tsl1, GPD1, GPD2, HOR2, RHR2 ectA, ectB, ectC, otsA, otsB, ProV, ProW, ProX, ProP, betA and betB.

11. The method of claim 1 wherein the microorganisms are selected from the group consisting of bacteria and yeast.

12. The method of claim 11 wherein the yeast is selected from the group consisting of Aspergillus nidulans, Aspergillus niger, Aspergillus oryzae, Candida albicans, Chrysosporium lucknowense, Fusarium graminearum, Fusarium venenatum, Kluyveromyces lactis, Neurospora crassa, Pichia angusta, Pichia finlandica, Pichia kodamae, Pichia membranaefaciens, Pichia methanolica, Pichia opuntiae, Pichia pastoris, Pichia pijperi, Pichia quercuum, Pichia salictaria, Pichia thermotolerans, Pichia trehalophila, Pichia stipitis, Streptomyces ambofaciens, Streptomyces aureofaciens, Streptomyces aureus, Saccharomyces bayanus, Saccharomyces boulardi, Saccharomyces cerevisiae, Streptomyces fungicidicus, Streptomyces griseochromogenes, Streptomyces griseus, Streptomyces lividans, Streptomyces olivogriseus, Streptomyces rameus, Streptomyces tanashiensis, Streptomyces vinaceus and Trichoderma reesei.

13. The method of claim 11 wherein the bacteria are selected from the group consisting of Bacillus subtilis, Bacillus amyloliquefacines, Brevibacterium ammoniagenes, Brevibacterium immariophilum, Clostridium beigerinckii, Clostridium acetobutylicum, Clostridium butylicum, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, Staphylococcus aureus and hydrocarbonoclastic bacteria.

14. The method of claim 13 wherein the hydrocarbonoclastic bacteria are selected from the group consisting of Alcanivorax, Cycloclasticus, Marinobacter, Neptunomonas, Oleiphilus, Oleispira and Thalassolitus.

15. The method of claim 1 further comprising the step of
(d) further purifying the organic component from the liquid phase rich in the organic component.

16. The method of claim 15 wherein step (d) comprises a distillation of the liquid phase rich in the organic component.

17. A method for producing an organic component comprising the steps of:
(A) culturing a microorganism in a fermentation medium to produce the organic component by said microorganism;
(B) recovering the organic product released by the microorganism into the fermentation medium from at least a portion of the fermentation medium by using the method for recovering an organic component from an aqueous solution as defined in claim 1.


SEQUENCE LISTING (15 sequences)

SEQ ID NO: 1 - protein, 495 aa, Saccharomyces cerevisiae
MTTDNAKAQLTSSSGGNIIVVSNRLPVTITKNSSTGQYEYAMSSGGLVTALEGLKKTYTFKWFGWPGLEIPDDEKDQVRKDLLEKFNAVPIFLSDEIADLHYNGFSNSILWPLFHYHPGEINFDENAWLAYNEANQTFTNEIAKTMNHNDLIWVHDYHLMLVPEMLRVKIHEKQLQNVKVGWFLHTPFPSSEIYRILPVRQEILKGVLSCDLVGFHTYDYARHFLSSVQRVLNVNTLPNGVEYQGRFVNVGAFPIGIDVDKFTDGLKKESVQKRIQQLKETFKGCKIIVGVDRLDYIKGVPQKLHAMEVFLNEHPEWRGKVVLVQVAVPSRGDVEEYQYLRSVVNELVGRINGQFGTVEFVPIHFMHKSIPFEELISLYAVSDVCLVSSTRDGMNLVSYEYIACQEEKKGSLILSEFTGAAQSLNGAIIVNPWNTDDLSDAINEALTLPDVKKEVNWEKLYKYISKYTSAFWGENFVHELYSTSSSSTSSSATKN

SEQ ID NO: 2 - protein, 896 aa, Saccharomyces cerevisiae
MTTTAQDNSPKKRQRIINCVTQLPYKIQLGESNDDWKISATTGNSALFSSLEYLQFDSTEYEQHVVGWTGEITRTERNLFTREAKEKPQDLDDDPLYLTKEQINGLTTTLQDHMKSDKEAKTDTTQTAPVTNNVHPVWLLRKNQSRWRNYAEKVIWPTFHYILNPSNEGEQEKNWWYDYVKFNEAYAQKIGEVYRKGDIIWIHDYYLLLLPQLLRMKFNDESIIIGYFHHAPWPSNEYFRCLPRRKQILDGLVGANRICFQNESFSRHFVSSCKRLLDATAKKSKNSSNSDQYQVSVYGGDVLVDSLPIGVNTTQILKDAFTKDIDSKVLSIKQAYQNKKIIIGRDRLDSVRGVVQKLRAFETFLAMYPEWRDQVVLIQVSSPTANRNSPQTIRLEQQVNELVNSINSEYGNLNFSPVQHYYMRIPKDVYLSLLRVADLCLITSVRDGMNTTALEYVTVKSHMSNFLCYGNPLILSEFSGSSNVLKDAIVVNPWDSVAVAKSINMALKLDKEEKSNLESKLWKEVPTIQDWTNKFLSSLKEQASSNDDMERKMTPALNRPVLLENYKQAKRRLFLFDYDGTLTPIVKDPAAAIPSARLYTILQKLCADPHNQIWIISGRDQKFLNKWLGGKLPQLGLSAEHGCFMKDVSCQDWVNLTEKVDMSWQVRVNEVMEEFTTRTPGSFIERKKVALTWHYRRTVPELGEFHAKELKEKLLSFTDDFDLEVMDGKANIEVRPRFVNKGEIVKRLVWHQHGKPQDMLKGISEKLPKDEMPDFVLCLGDDFTDEDMFRQLNTIETCWKEKYPDQKNQWGNYGFYPVTVGSASKKTVAKAHLTDPQQVLETLGLLVGDVSLFQSAGTVDLDSRGHVKNSESSLKSKLASKAYVMKRSASYTGAKV

SEQ ID NO: 3 - protein, 1054 aa, Saccharomyces cerevisiae
MTIIVASLFLPYTPQFEADVTNSDTAKLVESSMIKVDCNNQELSNNKQERSSSVTSASSHYIGLPQEAQINGEPLQRANVGSPATGVNYHNEMEMLSSEQFLEELTANATHAANSGIPPANNPVSSGSTAQRPSVEEFFSAPSARVCSPSQEASASSISASRSSAHHNDLSSSLMKNPNLSFDSHPPRVRSSSKSAVITPVSKSVPDVDPAVVDVAKVREEFQQQASLPSMKRVSGSTAGDSSIASSSSNLRYSQQFQDNFIEDTDSEDDIDSDLETDATKKYNVPKFGGYSNNAKLRASLMRNSYELFKHLPWTIVDSDKGNGSLKNAVNIAVAEKTVKEPVSWVGTMGIPTDELPHEVCHKISKKLEQDFSSFPVVTDDITFKGAYKNYAKQILWPTLHYQIPDNPNSKAFEDHSWDYYQKVNQKFSDRIVSVYKPGDTIWIHDYHLMLVPQMVREKLPKAKIGFFLHVSFPSSEVFRCLANRERILEGIIGANFVGFQTKEYKRHFLQTCNRLLAADVSNDEVKYHCNIVSVMYAPIGIDYYHLTSQLRNGSVLEWRQLIKERWRNKKLIVCRDQFDRIRGLQKKMLAYERFLIENPEYIEKVVLIQICIGKSSDPEYERQIMVVVDRINSLSSNISISQPVVFLHQDLDFAQYLALNCEADVFLVDALREGMNLTCHEFIVSSFEKNAPLLLSEFTGSSSVLKEGAILINPWDINHVAQSIKRSLEMSPEEKRRRWKKLFKSVIEHDSDNWITKCFEYINNAWESNQETSTVFNLAPEKFCADYKASKKHLFIFKISEPPTSRMLSLLSELSSNNIVYVLSSFTKNTFESLYNGVLNIGLIAENGAYVRVNGSWYNIVEELDWMKEVAKIFDEKVERLPGSYYKIADSMIRFHTENADDQDRVPTVIGEAITHINTLFDDRDIHAYVHKDIVFVQQTGLALAAAEFLMKFYNSGVSPTDNSRISLSRTSSSMSVGNNKKHFQNQVDFVCVSGSTSPIIEPLFKLVKQEVEKNNLKFGYTILYGSSRSTYAKEHINGVNELFTILHDLTAA

SEQ ID NO: 4 - protein, 1098 aa, Saccharomyces cerevisiae
MALIVASLFLPYQPQFELDTSLPENSQVDSSLVNIQAMANDQQQQRALSNNISQESLVAPAPEQGVPPAISRSATRSPSAFNRASSTTNTATLDDLVSSDIFMENLTANATTSHTPTSKTMLKPRKNGSVERFFSPSSNIPTDRIASPIQHEHDSGSRIASPIQQQQQDPTTNLLKNVNKSLLVHSLLNNTSQTSLEGPNNHIVTPKSRAGNRPTSAATSLVNRTKQGSASSGSSGSSAPPSIKRITPHLTASAAKQRPLLAKQPSNLKYSELADISSSETSSQHNESDPDDLTTAPDEEYVSDLEMDDAKQDYKVPKFGGYSNKSKLKKYALLRSSQELFSRLPWSIVPSIKGNGAMKNAINTAVLENIIPHRHVKWVGTVGIPTDEIPENILANISDSLKDKYDSYPVLTDDDTFKAAYKNYCKQILWPTLHYQIPDNPNSKAFEDHSWKFYRNLNQRFADAIVKIYKKGDTIWIHDYHLMLVPQMVRDVLPFAKIGFTLHVSFPSSEVFRCLAQREKILEGLTGADFVGFQTREYARHFLQTSNRLLMADVVHDEELKYNGRVVSVRFTPVGIDAFDLQSQLKDGSVMQWRQLIRERWQGKKLIVCRDQFDRIRGIHKKLLAYEKFLVENPEYVEKSTLIQICIGSSKDVELERQIMIVVDRINSLSTNISISQPVVFLHQDLDFSQYLALSSEADLFVVSSLREGMNLTCHEFIVCSEDKNAPLLLSEFTGSASLLNDGAIIINPWDTKNFSQAILKGLEMPFDKRRPQWKKLMKDIINNDSTNWIKTSLQDIHISWQFNQEGSKIFKLNTKTLMEDYQSSKKRMFVFNIAEPPSSRMISILNDMTSKGNIVYIMNSFPKPILENLYSRVQNIGLIAENGAYVSLNGVWYNIVDQVDWRNDVAKILEDKVERLPGSYYKINESMIKFHTENAEDQDRVASVIGDAITHINTVFDHRGIHAYVYKNVVSVQQVGLSLSAAQFLFRFYNSASDPLDTSSGQITNIQTPSQQNPSDQEQQPPASPTVSMNHIDFACVSGSSSPVLEPLFKLVNDEASEGQVKAGHAIVYGDATSTYAKEHVNGLNELFTIISRIIED

SEQ ID NO: 5 - protein, 391 aa, Saccharomyces cerevisiae
MSAAADRLNLTSGHLNAGRKRSSSSVSLKAAEKPFKVTVIGSGNWGTTIAKVVAENCKGYPEVFAPIVQMWVFEEEINGEKLTEIINTRHQNVKYLPGITLPDNLVANPDLIDSVKDVDIIVFNIPHQFLPRICSQLKGHVDSHVRAISCLKGFEVGAKGVQLLSSYITEELGIQCGALSGANIATEVAQEHWSETTVAYHIPKDFRGEGKDVDHKVLKALFHRPYFHVSVIEDVAGISICGALKNVVALGCGFVEGLGWGNNASAAIQRVGLGEIIRFGQMFFPESREETYYQESAGVADLITTCAGGRNVKVARLMATSGKDAWECEKELLNGQSAQGLITCKEVHEWLETCGSVEDFPLFEAVYQIVYNNYPMKNLPDMIEELDLHED

SEQ ID NO: 6 - protein, 440 aa, Saccharomyces cerevisiae
MLAVRRLTRYTFLKRTHPVLYTRRAYKILPSRSTFLRRSLLQTQLHSKMTAHTNIKQHKHCHEDHPIRRSDSAVSIVHLKRAPFKVTVIGSGNWGTTIAKVIAENTELHSHIFEPEVRMWVFDEKIGDENLTDIINTRHQNVKYLPNIDLPHNLVADPDLLHSIKGADILVFNIPHQFLPNIVKQLQGHVAPHVRAISCLKGFELGSKGVQLLSSYVTDELGIQCGALSGANLAPEVAKEHWSETTVAYQLPKDYQGDGKDVDHKILKLLFHRPYFHVNVIDDVAGISIAGALKNVVALACGFVEGMGWGNNASAAIQRLGLGEIIKFGRMFFPESKVETYYQESAGVADLITTCSGGRNVKVATYMAKTGKSALEAEKELLNGQSAQGIITCREVHEWLQTCELTQEFPLFEAVYQIVYNNVRMEDLPEMIEELDIDDE

SEQ ID NO: 7 - protein, 250 aa, Saccharomyces cerevisiae
MGLTTKPLSLKVNAALFDVDGTIIISQPAIAAFWRDFGKDKPYFDAEHVIQVSHGWRTFDAIAKFAPDFANEEYVNKLEAEIPVKYGEKSIEVPGAVKLCNALNALPKEKWAVATSGTRDMAQKWFEHLGIRRPKYFITANDVKQGKPHPEPYLKGRNGLGYPINEQDPSKSKVVVFEDAPAGIAAGKAAGCKIIGIATTFDLDFLKEKGCDIIVKNHESIRVGGYNAETDEVEFIFDDYLYAKDDLLKW

SEQ ID NO: 8 - protein, 250 aa, Saccharomyces cerevisiae
MPLTTKPLSLKINAALFDVDGTIIISQPAIAAFWRDFGKDKPYFDAEHVIHISHGWRTYDAIAKFAPDFADEEYVNKLEGEIPEKYGEHSIEVPGAVKLCNALNALPKEKWAVATSGTRDMAKKWFDILKIKRPEYFITANDVKQGKPHPEPYLKGRNGLGFPINEQDPSKSKVVVFEDAPAGIAAGKAAGCKIVGIATTFDLDFLKEKGCDIIVKNHESIRVGEYNAETDEVELIFDDYLYAKDDLLKW

SEQ ID NO: 9 - protein, 172 aa, Marinococcus halophilus
METKMTGTNGSVDSIVFDKPTVEDGADMWELVKNSTLDLNSSYKYIMMCEFFAETCVVAKENDELVGFVTAFIPPEKQDTVFVWQVGVDTSQRGKGLASRLLNALLERDVCENVLYLEATITPSNEASQALFKKLAQKRETEVTVSECFTEDLFPDDEHEEELTFRIGPFTK

SEQ ID NO: 10 - protein, 427 aa, Marinococcus halophilus
MMQNDLSVFNEYESEVRSYVRGFPTVFHQAKGYKLWDLDGKEYVDFFSGAGALNYGHNDENMKQKLLTYIQEDGVTHSLDMATKAKGEFIDAFQNIILKPRNMDYKIMFPGPTGANSVESALKLARKVTGRTNVVSFTNGFHGMTIGALSVTGNKFKRNGAGMPLSNTSTLPYDQFLKESNNSIEYIENFLDNGGSGLDKPAAFIVETVQGEGGLNAASSEWLRSIEKICRERDIKLILDDVQAGVGRTGTFFSFEPAGIKPDFVCLSKSIGGNGSPLAITLVAPEYDKFAPGEHNGTFRGNNFAFVTGTEALNYWKDDRLEKNVQEKSERITSFLDDMIKKHPEMKGVRKGRGFMQGIMSPIEDLADNIAGRCFEHGLIMETAGAEDEVFKLFPPITIDDEGLERGLSILQQAIEEVTAESNLVAK

SEQ ID NO: 11 - protein, 129 aa, Marinococcus halophilus
MKVIKLEDLLGTEREVDDGNWVSRRFIMKDDNMGYSVNDTIIRAGTETHIWYQNHLETVYCIEGDGEIETLSDNKVYQLEPGVLYALDKNDEHMLRGGSKDMRMVCVFNPPLSGREVHDENGVYPADLD

SEQ ID NO: 12 - protein, 474 aa, Escherichia coli
MSRLVVVSNRIAPPDEHAASAGGLAVGILGALKAAGGLWFGWSGETGNEDQPLKKVKKGNITWASFNLSEQDLDEYYNQFSNAVLWPAFHYRLDLVQFQRPAWDGYLRVNALLADKLLPLLQDDDIIWIHDYHLLPFAHELRKRGVNNRIGFFLHIPFPTPEIFNALPTYDTLLEQLCDYDLLGFQTENDRLAFLDCLSNLTRVTTRSAKSHTAWGKAFRTEVYPIGIEPKEIAKQAAGPLPPKLAQLKAELKNVQNIFSVERLDYSKGLPERFLAYEALLEKYPQHHGKIRYTQIAPTSRGDVQAYQDIRHQLENEAGRINGKYGQLGWTPLYYLNQHFDRKLLMKIFRYSDVGLVTPLRDGMNLVAKEYVAAQDPANPGVLVLSQFAGAANELTSALIVNPYDRDEVAAALDRALTMSLAERISRHAEMLDVIVKNDINHWQECFISDLKQIVPRSAESQQRDKVATFPKLA

SEQ ID NO: 13 - protein, 266 aa, Escherichia coli
MTEPLTETPELSAKYAWFFDLDGTLAEIKPHPDQVVVPDNILQGLQLLATASDGALALISGRSMVELDALAKPYRFPLAGVHGAERRDINGKTHIVHLPDAIARDISVQLHTVIAQYPGAELEAKGMAFALHYRQAPQHEDALMTLAQRITQIWPQMALQQGKCVVEIKPRGTSKGEAIAAFMQEAPFIGRTPVFLGDDLTDESGFAVVNRLGGMSVKIGTGATQASWRLAGVPDVWSWLEMITTALQQKRENNRSDDYESFSRSI

SEQ ID NO: 14 - protein, 500 aa, Escherichia coli
MLKRKKVKPITLRDVTIIDDGKLRKAITAASLGNAMEWFDFGVYGFVAYALGKVFFPGADPSVQMVAALATFSVPFLIRPLGGLFFGMLGDKYGRQKILAITIVIMSISTFCIGLIPSYDTIGIWAPILLLICKMAQGFSVGGEYTGASIFVAEYSPDRKRGFMGSWLDFGSIAGFVLGAGVVVLISTIVGEANFLDWGWRIPFFIALPLGIIGLYLRHALEETPAFQQHVDKLEQGDREGLQDGPKVSFKEIATKYWRSLLTCIGLVIATNVTYYMLLTYMPSYLSHNLHYSEDHGVLIIIAIMIGMLFVQPVMGLLSDRFGRRPFVLLGSVALFVLAIPAFILINSNVIGLIFAGLLMLAVILNCFTGVMASTLPAMFPTHIRYSALAAAFNISVLVAGLTPTLAAWLVESSQNLMMPAYYLMVVAVVGLITGVTMKETANRPLKGATPAASDIQEAKEILVEHYDNIEQKIDDIDHEIADLQAKRTRLVQQHPRIDE

SEQ ID NO: 15 - protein, 556 aa, Escherichia coli
MQFDYIIIGAGSAGNVLATRLTEDPNTSVLLLEAGGPDYRFDFRTQMPAALAFPLQGKRYNWAYETEPEPFMNNRRMECGRGKGLGGSSLINGMCYIRGNALDLDNWAQEPGLENWSYLDCLPYYRKAETRDMGENDYHGGDGPVSVTTSKPGVNPLFEAMIEAGVQAGYPRTDDLNGYQQEGFGPMDRTVTPQGRRASTARGYLDQAKSRPNLTIRTHAMTDHIIFDGKRAVGVEWLEGDSTIPTRATANKEVLLCAGAIASPQILQRSGVGNAELLAEFDIPLVHELPGVGENLQDHLEMYLQYECKEPVSLYPALQWWNQPKIGAEWLFGGTGVGASNHFEAGGFIRSREEFAWPNIQYHFLPVAINYNGSNAVKEHGFQCHVGSMRSPSRGHVRIKSRDPHQHPAILFNYMSHEQDWQEFRDAIRITREIMHQPALDQYRGREISPGVECQTDEQLDEFVRNHAETAFHPCGTCKMGYDEMSVVDGEGRVHGLEGLRVVDASIMPQIITGNLNATTIMIGEKIADMIRGQEALPRSTAGYFVANGMPVRAKK